(19) United States
(12) Patent Application Publication
(10) Pub. No.: US 2024/0100291 A1
(43) Pub. Date: Mar. 28, 2024

(54) DYNAMIC COMPLIANCE PATIENT CIRCUIT

(71) Applicant: Covidien LP, Mansfield, MA (US)

(72) Inventors: Amand V. Kasimatis, Carlsbad, CA (US); Patrick J. Nellis, Georgetown (CA); Matthew J. Phillips, Carlsbad, CA (US)

(73) Assignee: Covidien LP, Mansfield, MA (US)

(21) Appl. No.: 18/466,360

(22) Filed: Sep. 13, 2023

Related U.S. Application Data
(60) Provisional application No. 63/410,309, filed on Sep. 27, 2022.

(51) Int. Cl. A61M 16/20; A61M 16/00

(52) U.S. Cl. CPC A61M 16/204; A61M 16/0003; A61M 16/024

(57) ABSTRACT

Methods and systems for dynamically adjusting the compliance of a patient circuit. In an example, the technology relates to a medical ventilation system that includes a dynamic compliance circuit. The dynamic compliance circuit includes an inspiratory tube extending from an inhalation port of a medical ventilator, the inspiratory tube defining an inner lumen for carrying breathing gases from the inhalation port towards a patient; and a compliance adjustment covering coupled to the inspiratory tube, wherein adjustments to the compliance adjustment covering alter an effective compliance of the inspiratory tube. The system may also include a processor; and memory storing instructions that, when executed by the processor, cause the system to perform operations including, based a type of ventilation mode of the ventilator, causing an adjustment to the compliance adjustment covering to alter the effective compliance of the inspiratory tube.

### CROSS-REFERENCE TO RELATED APPLICATIONS

This application claims the benefit of U.S. Provisional Application No. 63/410,309 filed Sep. 27, 2022, entitled “Dynamic Compliance Patient Circuit,” which is incorporated herein by reference in its entirety.

### INTRODUCTION

Medical ventilator systems have long been used to provide ventilatory and supplemental oxygen support to patients. These ventilators typically comprise a connection for pressurized gas (air, oxygen) that is delivered to the patient through a conduit or tubing. Different patients may require different ventilation strategies, and modern ventilators may provide may different modes or settings. For example, several different ventilator modes or settings have been created to provide better ventilation for patients in different scenarios, such as mandatory ventilation modes, spontaneous ventilation modes, and assist-control ventilation modes. In some examples, a patient may be provided with ventilation under one mode or setting, and then a second or subsequent mode of ventilation may be utilized where a condition of the patient has changed.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Among other things, aspects of the present disclosure relates to methods and systems for dynamically adjusting the compliance of a patient circuit. In an aspect, the technology relates to a medical ventilation system that includes a dynamic compliance circuit. The dynamic compliance circuit includes an inspiratory tube extending from an inhalation port of a medical ventilator, the inspiratory tube defining an inner lumen for carrying breathing gases from the inhalation port towards a patient; and a compliance adjustment covering coupled to the inspiratory tube, wherein adjustments to the compliance adjustment covering alter an effective compliance of the inspiratory tube. The system may also include a processor; and memory storing instructions that, when executed by the processor, cause the system to perform operations including, based a type of ventilation mode of the ventilator, causing an adjustment to the compliance adjustment covering to alter the effective compliance of the inspiratory tube.

In an example, the compliance adjustment covering includes a mesh capable of being tightened by pulling tightening extensions extending from the mesh. In another example, causing the adjustment to the compliance adjustment covering includes causing the tightening extensions to be pulled. In yet another example, the compliance adjustment covering includes an outer tube defining an outer lumen between the outer tube and inspiratory tube, the outer lumen capable of receiving a compliance-adjusting pressurized gas. In a further example, causing the adjustment to the compliance adjustment covering includes causing a delivery of the compliance-adjusting pressurized gas to increase the effective compliance of the inspiratory tube. In still another example, the outer lumen is at least partially filled with a non-Newtonian fluid in contact with an outer surface of the inspiratory tube. In still yet another example, the compliance adjustment covering includes an outer tube spirally wrapped around the inspiratory tube, the outer tube defining an outer lumen between the outer tube and inspiratory tube, the outer lumen capable of receiving a compliance-adjusting pressurized gas.

In another aspect, the technology relates to a dynamic compliance circuit for medical ventilation that includes an inspiratory tube extending from an inhalation port of a medical ventilator, the inspiratory tube having an inner lumen for carrying breathing gases from the inhalation port towards a patient; and a compliance adjustment covering coupled to the inspiratory tube, wherein adjustments to the compliance adjustment covering alter an effective compliance of the inspiratory tube.

In an example, the compliance adjustment covering includes a mesh capable of being tightened by pulling tightening extensions extending from the mesh. In another example, the compliance adjustment covering includes an outer tube defining an outer lumen between the outer tube and inspiratory tube, the outer lumen capable of receiving a compliance-adjusting pressurized gas. In yet another example, the compliance adjustment covering includes an outer tube spirally wrapped around the inspiratory tube, the outer tube defining an outer lumen between the outer tube and inspiratory tube, the outer lumen capable of receiving a compliance-adjusting pressurized gas.

In another aspect, the technology relates to a method for controlling an effective compliance of an inspiratory tube. The method includes detecting at a first time, based on a gas property sensor, a first breathing gas property of breathing gases flowing through the inspiratory tube; based on the first breathing gas property, causing a first adjustment to the compliance adjustment covering to increase the effective compliance of the inspiratory tube; detecting at a second time, based on the gas property sensor, a second breathing gas property of breathing gases flowing through the inspiratory tube; and based on the second breathing gas property, causing a second adjustment to the compliance adjustment covering to decrease the effective compliance of the inspiratory tube.

In an example, the first breathing gas property is a first breathing gas pressure; the second breathing gas property is a second breathing gas pressure; and the first breathing gas pressure is higher than the second breathing gas pressure. In another example, the first adjustment and the second adjustment are caused within a time period of less than 5 seconds. In still another example, the compliance adjustment covering includes an outer tube defining an outer lumen, the outer lumen capable of receiving a compliance-adjusting pressurized gas; and causing a first adjustment to the compliance adjustment covering includes increasing the pressure of the compliance-adjusting pressurized gas.

It is to be understood that both the foregoing general description and the following Detailed Description are explanatory and are intended to provide further aspects and examples of the disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1A is a diagram illustrating an example of a medical ventilator and compliance adjustment sleeve connected to a human patient.

FIG. 1B depicts the ventilation system where a compliance adjustment sleeve is formed as an outer tube.

FIG. 1C depicts the ventilation system where a compliance adjustment sleeve is formed as a mesh or spiral cover.

FIG. 2 is a cross-sectional view of a dynamic compliance circuit.

FIG. 3 is a cross-sectional view of a dynamic compliance circuit with a non-Newtonian fluid.

FIG. 4 is a perspective view of a dynamic compliance circuit including a compliance adjustment sleeve that includes a mesh cover.

FIG. 5 is a perspective view of a dynamic compliance circuit with longitudinal compliance restrictors.

FIG. 6 is a perspective view of an example dynamic compliance circuit with a spirally wrapped outer tube.

FIG. 7 depicts an example method for dynamically adjusting an effective compliance of an inner tube.

FIG. 8 depicts another example method for dynamically adjusting an effective compliance of an inner tube.

FIG. 9 depicts an example method for dynamically adjusting an effective compliance of an inner tube.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

Medical ventilators are used to provide breathing gases to patients who are otherwise unable to breathe sufficiently. In modern medical facilities, pressurized air and oxygen sources are often available from wall outlets, tanks, or other sources of pressurized gases. Accordingly, ventilators may provide pressure regulating valves (or regulators) connected to centralized sources of pressurized air and pressurized oxygen. The regulating valves function to regulate flow so that respiratory gases having a desired concentration are supplied to the patient at desired pressures and flow rates. Further, as each patient may require a different ventilation strategy, modern ventilators may be customized for the particular needs of an individual patient.

As briefly discussed above, different types of ventilation modes may be used to provide medical ventilation to a patient. Depending on the type of mode that is selected, different types of patient circuits (e.g., tubing) may be used to carry the breathing gases from the ventilator to the patient. For instance, the pressure and/or flow provided by the ventilator may cause the tubing of the patient circuit to stretch or expand where the pressures of the breathing gases are high. As an example, in the case of a ventilation mode such as high-frequency oscillatory ventilation (HFOV), the high pressures and frequencies of the breathing gases that are delivered causes the tubing to expand. Some patient circuit compliance (e.g., stretching or expanding) is to be expected. However, as the pressure of the gas delivered to the patient circuit increases (as with ventilation modes such as HFOV), some patient circuits expand beyond a desirable amount and create a larger volume within the tubing, which can lead to reduced effectiveness of the ventilation. Therefore, the intended benefits of HFOV may be lost due to tubing that is too compliant.

Currently, to avoid this type of undesirable expansion, a different type of patient circuit is used for high-pressure or frequency modes of ventilation, such as a HFOV. For instance, the patient circuit for HFOV has a stiffer compliance to reduce expansion and help deliver the intended ventilation to the patient. Due to the use of a different patient circuit, changing the ventilation mode causes respiratory therapists to stop patient ventilation, disconnect the patient circuit, and reconnect a different patient circuit in order to switch ventilation modes to mode such as HFOV. This potentially puts the patient at risk from the disruption in ventilatory support. The patient is also potentially at a higher risk of infection as parts of the ventilator system are left open while the patient circuit is changed. Disconnection of the patient circuit may also cause pathogens within the patient circuit to be released into the room air. For at least these reasons, a system and method for a dynamic compliance patient circuit that adapts to changes in ventilation mode, and therefore does not need to be replaced, is needed.

Among other things, the systems and methods disclosed herein address these circumstances by providing a dynamic compliance patient circuit that is adaptive at least to a traditional mode of ventilation and a high-pressure mode of ventilation, such as HFOV. The dynamic compliance circuit is capable of adjusting its compliance during ventilation. Accordingly, when switching from one ventilator mode to another, the compliance of the circuit is dynamically adjusted, which prevents the need to remove one patient circuit and replace it with another. The compliance adjustment may be performed automatically by the ventilator and/or other control system, or the change in compliance may be performed manually in some examples.

Examples of dynamic compliance circuits include a compliance adjustment covering that is adaptive to a ventilation mode. The compliance adjustment covering wraps around at least a portion of an inner tube that carries the breathing gases. The compliance adjustment covering may be implemented in several different manners, including an outer tubing with a lumen that can be selectively pressurized to adjust the effective compliance of an inner tubing of the patient circuit. The compliance adjustment covering may also be formed as an adjustable mesh or sleeve that can be tightened to adjust the compliance of the inner tubing of the patient circuit. Additional details and examples of dynamic compliance patient circuits, along with methods for controlling such circuits and systems incorporating such circuits, are discussed below.

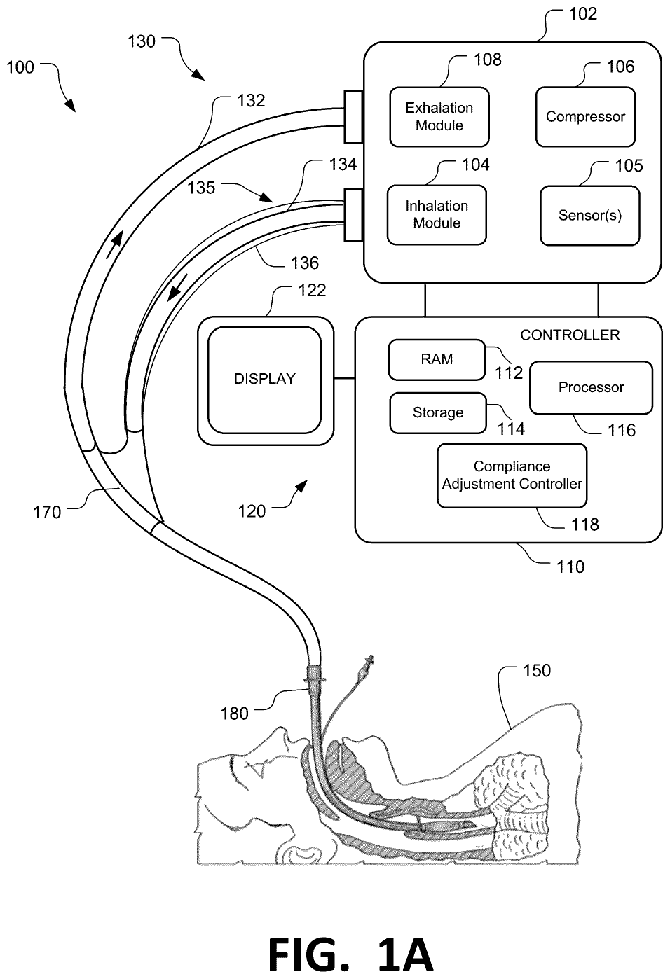

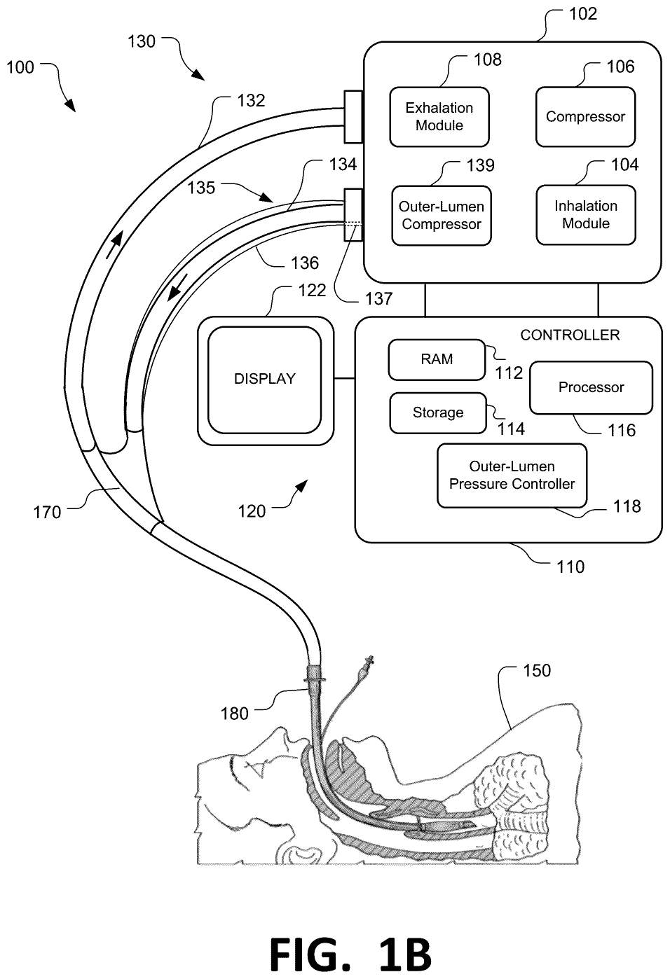

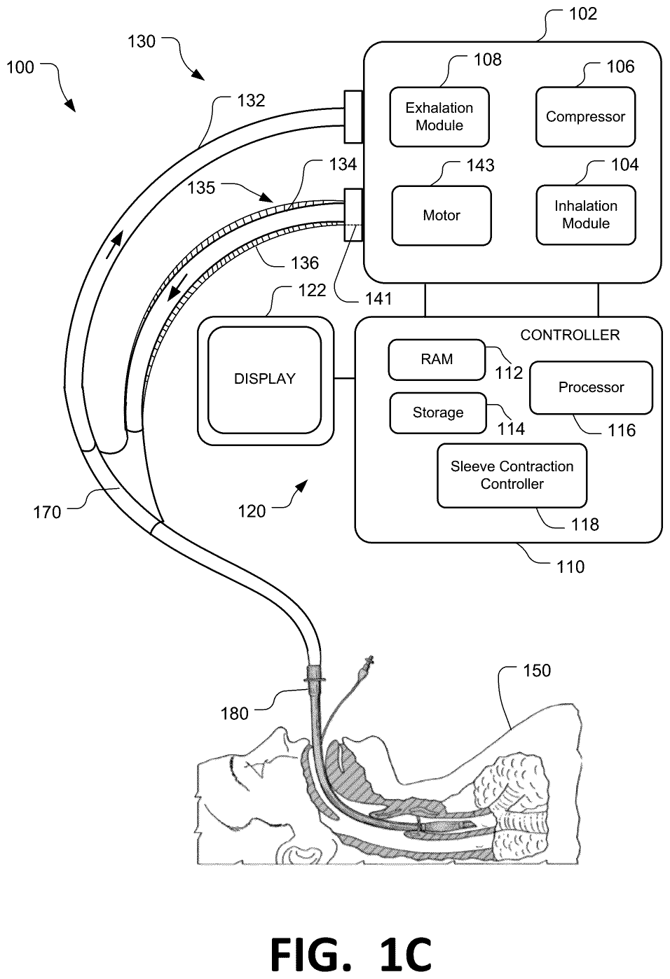

FIG. 1 is a diagram illustrating an example of a medical ventilation system 100 connected to a human patient 150. The ventilator 100 provides positive pressure ventilation to the patient 150. Ventilation system 100 includes a pneumatic system 102 (also referred to as a pressure generating system 102) for circulating breathing gases to and from patient 150 via the ventilation tubing system 130, which couples the patient 150 to the pneumatic system 102 via an invasive (e.g., endotracheal tube, as shown) or a non-invasive (e.g., nasal mask) patient interface 180.

Ventilation tubing system 130 may be a two-limb (shown) or a one-limb circuit for carrying gases to and from the patient 150. In a two-limb example, a fitting, typically referred to as a “wye-fitting” 170, may be provided to couple a patient interface 180 to an inhalation limb 134 and an exhalation limb 132 of the ventilation tubing system 130. The tubing system 130 may also be referred to as a patient circuit 130.

In the example depicted, the inhalation limb 134 is a dynamic compliance circuit 135 that includes an inner tubing and a compliance adjustment sleeve 136. In examples, the inner tubing of the inhalation limb 134 defines an inner lumen that carries breathing gases to human patient 150 from pneumatic system 102. In various implementations, the compliance adjustment covering or sleeve 136 is coupled over or around the inner tubing of the inhalation limb 134. The compliance adjustment sleeve 136 may expand, stretch, contract, or otherwise adjust based on the pressure of the gas delivered from pneumatic system 102 to human patient 150, based on a ventilation mode set using controller 110, and/or manual adjustments to the compliance adjustment sleeve 136. For example, adjustments to the compliance adjustment sleeve 136 alter an effective compliance of the inspiratory tube within inhalation limb 134.

Pneumatic system 102 may have a variety of configurations. In the present example, system 102 includes an exhalation module 108 coupled with the exhalation limb 132 and an inhalation module 104 coupled with the inhalation limb 134. Compressor 106, or other source(s) of pressurized gases (e.g., air, oxygen, and/or helium), is coupled with inhalation module 104 to provide a gas source for ventilatory support via inhalation limb 134. The pneumatic system 102 may include a variety of other components, including mixing modules, valves, tubing, accumulators, filters, etc. For instance, the pneumatic system 102 may include sensors 105, which may be internal or external sensors to the ventilator (and may be communicatively coupled, or capable of communicating, with the ventilator). The sensors 105 may include gas property sensors that measure gas properties such as pressure (e.g., via a pressure sensor) and/or flow (e.g., via a flow sensor) of the breathing gases flowing within tubing system 130.

Controller 110 is operatively coupled with pneumatic system 102, signal measurement and acquisition systems, and an operator interface 120 that may enable an operator to interact with the ventilator 100 (e.g., change ventilation settings, select operational modes, view monitored parameters, etc.). Controller 110 may include memory 112, one or more processors 116, storage 114, and/or other components of the type found in command-and-control computing devices. In the depicted example, operator interface 120 includes a display 122 that may be touch-sensitive and/or voice-activated, enabling the display 122 to serve both as an input and output device. The combination of the pneumatic system 102, the controller 110, and/or the display 122 may be considered as a medical ventilator.

The memory 112 includes non-transitory, computer-readable storage media that stores software that is executed by the processor 116 and which controls the operation of the ventilator 100. In an example, the memory 112 includes one or more solid-state storage devices 114 such as flash memory chips. In an alternative example, the memory 112 may be mass storage connected to the processor 116 through a mass storage controller (not shown) and a communications bus (not shown). Although the description of computer-readable media contained herein refers to a solid-state storage, the computer-readable storage media may be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer. The memory 112 stores instructions that, when executed, cause the system to perform the operations discussed herein.

The controller 110 may also include a compliance-adjustment controller 118. The compliance-adjustment controller 118 adjusts the compliance adjustment sleeve 136. The compliance-adjustment controller 118 may cause adjustment of the effective compliance of the dynamic compliance circuit 135 in various manners, such as increasing pressure within an outer lumen of the dynamic compliance circuit 135 and/or contracting the compliance-adjustment sleeve 136. The compliance-adjustment controller 118 may receive indications of a particular ventilation mode from other components of the controller 110. For instance, if a ventilation mode is selected or changed, the compliance-adjustment controller 118 may receive an indication of such a change. Alternatively or additionally, the compliance-adjustment controller 118 may receive gas properties of the breathing gases flowing through the tubing system 130 as measured or sensed by the sensors 105. Based on the detected or received ventilation mode and/or gas properties, the compliance-adjustment controller 118 adjusts the dynamic compliance circuit 135 to increase or decrease the effective compliance of the inner tubing carrying the breathing gases, such as the inhalation limb 134.

While the compliance-adjustment controller 118 is depicted as being in the ventilator in the present example, in other examples the compliance-adjustment controller 118 may be within a standalone control system or integrated into the control system of another device. For instance, the compliance-adjustment controller 118 may be integrated into a control system for inflating a cuff of an endotracheal tube.

FIG. 1B depicts the ventilation system 100 where the compliance adjustment sleeve 136 is formed as an outer tube that forms an outer lumen between the outer tube and the inner tube of the inhalation limb 134, as discussed further herein, and the compliance-adjustment controller operates as an outer-lumen pressure controller 118 to control pressure within the outer lumen. For example, the pneumatic system 102 may include an outer-lumen compressor 139 and an outer-lumen port 137. The outer-lumen port 137 connects to the outer lumen of the dynamic compliance circuit 135 to allow for gas (e.g., air) to be provided into the outer lumen from the outer-lumen compressor 139. The compliance-adjustment controller 118 controls outer-lumen compressor 139 to adjust the pressure of the gas within the outer lumen. For example, to increase the pressure of gas within the outer lumen, the compliance-adjustment controller 118 causes additional gas to be delivered into the outer lumen by activating the outer-lumen compressor 139. Conversely, to reduce the pressure in the outer lumen, the outer-lumen port 137 may be opened (e.g., a valve controlling the port) to allow for gas to escape the outer lumen, which reduces the pressure within the outer lumen.

The pressure of the gas within the outer lumen may be adjusted based on a type of mode of ventilation. For instance, the pressure of the gas in the outer lumen may be adjusted to be a first pressure for a first type of ventilation mode (e.g., a lower-pressure ventilation mode) and may be adjusted to be a second, higher pressure for a second type of ventilation mode (e.g., a higher-pressure ventilation mode such as HFOV). In such examples, the outer-lumen pressure controller 118 detects or receives an indication that the ventilation mode has been changed. The pressure of the gas in the outer lumen may also be adjusted based on the gas properties, such as pressure or flow, of the breathing gases delivered flowing through the breathing circuit 130.

FIG. 1C depicts the ventilation system 100 where the compliance adjustment sleeve 136 is formed as a mesh cover that can be contracted or released mechanically, such as through tightening extensions in the form of pull wires. In such an example, the compliance-adjustment controller 118 is formed as a sleeve contraction controller 118 and the pneumatic system 102 includes a motor 143 for contracting or releasing extensions of the mesh cover, as discussed in further detail below. The pneumatic system 102 may include an extension adapter 141 at or near the inspiratory port. One end of the extension adapter connects or couples to the extension(s) of the cover, and the other end of the extension adapter couples to the motor 143 such that the motor is capable of contracting (e.g., pulling) the extension(s). For example, pulling the extension causes the mesh cover to contract or constrict around the inner tubing, such as the inspiratory limb 134. As the mesh cover contracts or constricts around the inner tubing, the effective compliance of the inner tubing is reduced (e.g., mesh cover reduces the amount the inner tubing can expand). Conversely, releasing the extension allows for the mesh cover to expand and provide less resistance to the expansion of the inner tubing.

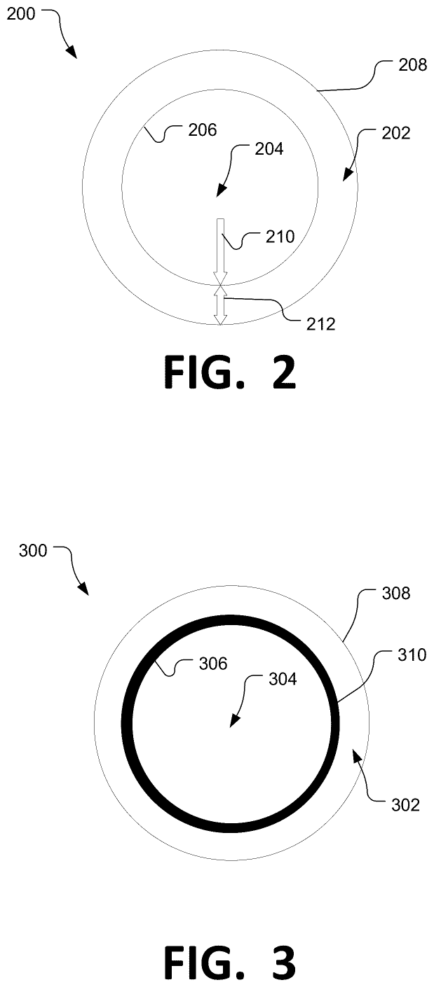

FIG. 2 is a cross-sectional view of an example dynamic compliance circuit 200. As described above, the dynamic compliance circuit 200 may be included within an inhalation limb, such as inhalation limb 134 as shown in FIG. 1. The dynamic compliance circuit 200 has an inner tube 206 and an outer tube 208. The outer tube 206 is one example of a compliance adjustment sleeve discussed above with respect to FIG. 1.

An inner lumen 204 is defined within the inner tube 206, as illustrated. Where the dynamic compliance circuit 200 forms a portion of the inhalation limb, the inner lumen 204 carries breathing gases from an inhalation port on a pneumatic system towards a human patient, such as pneumatic system 102 and human patient 150 as shown in FIG. 1. An outer lumen 202 is formed between the inner tube 206 and the outer tube 208. In various implementations, outer lumen 202 may be filled with a fluid, such as gas (e.g., atmospheric gas) or a liquid, and the pressure of that fluid may be dynamically controlled. To distinguish the gases or fluids in the outer lumen 202 from the breathing gases in the inner lumen 204, the fluids or gases in the outer lumen 202 may be referred to as compliance-adjustment pressurized gases or fluids.

When breathing gases flow through the inner lumen 204, the pressure of those breathing gases exerts an outward force on the inner tube 206 as indicated by the inner pressure indicator 210. This outward force causes the inner tube 206 to expand radially outward. The amount that the inner tube 206 expands depends on the compliance of the inner tube 206. For instance, an inner tube 206 with a high compliance expands more than an inner tube 206 with a low compliance. As discussed above, during high-pressure ventilation modes, too much expansion of the inner tube 206 may produce unfavorable results. However, using an inner tube 206 that has a low compliance (e.g., a stiff tube), is difficult to manipulate by a patient or a respiratory therapist in connecting the patient to the ventilator.

With the present technology, the pressure of gases in the outer lumen 202 may be controlled to provide an inward force against the inner tube 206, as indicated by the outer pressure indicator 212. The gas pressure within outer lumen 202 also provides an outward force against the outer tube 208. As the pressure of the gas in the outer lumen 202 is increased, the ability of the inner tube 206 to expand, due to the breathing gases, is reduced. Thus, while the physical composition of the inner tube 206 does not change, the effective compliance of the inner tube 206 is reduced as the pressure in the outer lumen 202 increases. Accordingly, the present technology may control the pressure of the gases in the outer lumen 202 to offset or counteract the expansion forces of the breathing gases in the inner lumen 204.

As an example, the pressure of the breathing gases within the inner lumen 204 may be determined or detected, such as through the use of a pressure sensor. Based on the pressure of the breathing gases, the pressure of the fluid in the outer lumen 202 is dynamically increased based on increases in pressure of the breathing gases. In some examples, the pressure of the fluid in the outer lumen 202 is increased to be at least 50%, 80%, 90%, 100%, 200%, 300%, or the like, of the pressure of the breathing gases within the inner lumen 204. The pressure of the fluid in the outer lumen 202 may also be adjusted based on the type of ventilation mode that is being implemented. For instance, in some ventilation modes, the pressure of the fluid in the outer lumen 202 may be unchanged or left at atmospheric pressure. In other ventilation modes, such as HFOV, the pressure of the fluid in the outer lumen 202 is increased to offset the expansion forces from the pressure of the breathing gases.

FIG. 3 is a cross-sectional view of another example dynamic compliance circuit 300 with a non-Newtonian fluid 310. The dynamic compliance circuit 300 is similar to the dynamic compliance circuit 200 depicted in FIG. 2 and discussed above. For instance, the dynamic compliance circuit 300 has an inner tube 306 and an outer tube 308, an inner lumen 304 is defined within the inner tube 306, and an outer lumen 202 is formed between the inner tube 306 and the outer tube 308.

In the dynamic compliance circuit 300, however, the outer lumen 302 is at least partially filled with a non-Newtonian fluid 310 that in contact with the outer surface of the inner tube 306 of the dynamic compliance circuit 300. In some examples, the non-Newtonian fluid may fill at least 30%, 50%, or 80% of the outer lumen 302.

Non-Newtonian fluid 310 may resist sheer, stress, or pressure to which it is subjected. In such implementations, when breathing gases flow through inner lumen 304, non-Newtonian fluid 310 may resist the expansion of the inner tube 306, thereby allowing the patient circuit to have a dynamic (e.g. adaptable) compliance with no need to change out the patient circuit when the ventilation mode is changed. Non-Newtonian fluid 310 may resist frequency-based motions, though it may still allow the dynamic compliance circuit 300 to bend based on movements from the patient or a respiratory therapist when configuring or moving the patient circuit. In some examples, non-Newtonian fluid 310 may have dilatant and/or rheopectic properties. The pressure of gases or other fluids in the outer lumen 302 may also be controlled as discussed above.

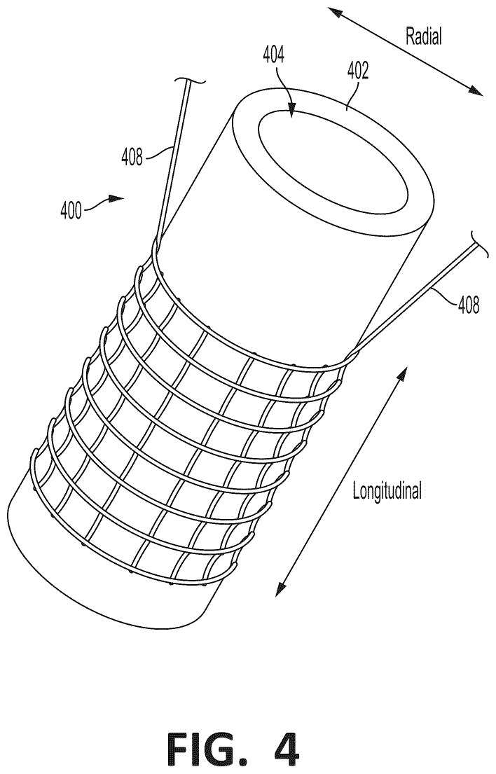

FIG. 4 is a perspective view of a dynamic compliance circuit 400 including a compliance adjustment covering that includes a mesh cover 406. Dynamic compliance circuit 400 includes a tube 402 that defines an inner lumen 404. As described above, inner lumen 404 is configured to carry breathing gases from the inhalation port towards a human patient.

The compliance adjustment covering, including a mesh cover 406, is coupled to tube 402, which may be an inspiratory tube. Adjustments to the mesh cover 406 alter an effective compliance the tube 402. In examples, tube 402 may experience radial and/or longitudinal expansion as breathing gasses flow through inner lumen 404, as discussed above. To restrict such expansion, tightening extensions 408 of mesh cover 406 may be pulled to constrict or tighten the mesh cover 406 around the tube 402. In examples, the tightening extensions 408 may be strings, wires, additional tubing, threads, or the like. The tightening extensions 408 of the mesh cover 406 may be tightened by mechanical means controlled by a controller. For instance, as discussed above with reference to FIG. 1, a motor may be controlled to tighten and loosen the tightening extensions 408. The tightening and/or loosening of the tightening extensions may be based on pressure of the breathing gases and/or the type of ventilator mode, as discussed herein.

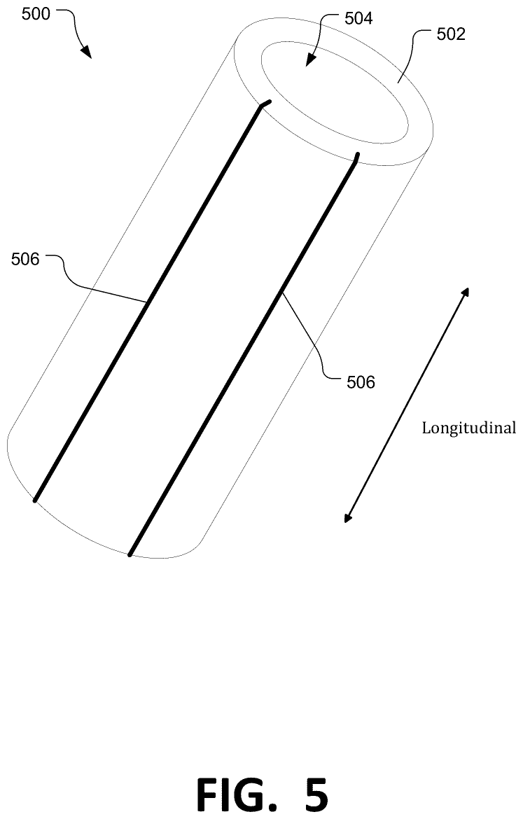

FIG. 5 is a perspective view of a dynamic compliance circuit 500 with longitudinal compliance restrictors 506. The dynamic compliance circuit 500 may be similar to those discussed above. For instance, the dynamic compliance circuit 500 includes a tube 502, which may be an inspiratory tube, that defines an inner lumen 504 that carries breathing gases.

As discussed above, the pressures of the breathing gases may cause a radial expansion as well as a longitudinal expansion. The mesh cover and outer lumen discussed above may primarily restrict radial expansion. To restrict longitudinal expansion, one or more longitudinal compliance restrictors 506 may be incorporated into the dynamic compliance circuit 500.

The longitudinal compliance restrictors 506 extend longitudinally across the tube 502 and are coupled or integrated into the tube 502. For instance, the longitudinal compliance restrictors 506 may be integrated into the wall of the tube 502 or coupled to the exterior surface of the tube 502. The longitudinal compliance restrictors 506 are more rigid, or have a lower compliance, than the tube 502 and resist expansion in the longitudinal direction. In some examples, the longitudinal compliance restrictors 506 may be formed of a wire or other elongate element that resists longitudinal expansion. Multiple longitudinal compliance restrictors 506 may also be provided on the tube 502. For instance, at least 2, 3, 4, or more longitudinal compliance restrictors 506 may be incorporated. The longitudinal compliance restrictors 506 may be used in combination with the other types of compliance adjustment sleeves or covers discussed herein.

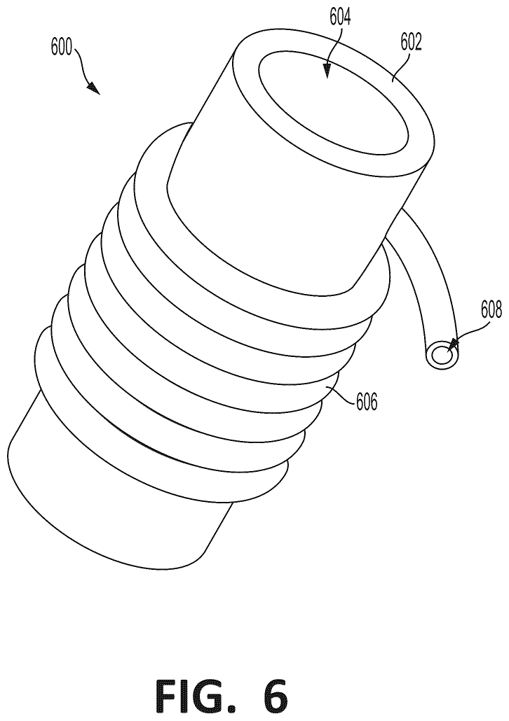

FIG. 6 is a perspective view of an example dynamic compliance circuit 600 with a spirally wrapped outer tube 606. Similar also to the other dynamic compliance circuits discussed herein, the dynamic compliance circuit 600 includes an inner tube 602, which may be an inspiratory tube, that defines an inner lumen 604 that carries breathing gases. The compliance adjustment covering in the dynamic compliance circuit 600 is in the form of an outer tube 606 spirally wrapped around inner tube 602.

The outer tube 606 defines an outer lumen 608 that also spirally wraps around the outside of the inner tube 602. The outer lumen 608 be defined on all sides by the outer tube 606. In other examples, the outer tube 606 may share a common wall with the exterior of the inner tube 602. In such examples, the outer lumen 608 may be defined by the inner surface of the outer tube 606 and the exterior surface of the inner tube 602. The outer tube 606 may be coupled to the inner tube 602 in a variety of manners. For instance, the outer tube 606 may be bonded or adhered to the inner tube 602. In other examples, the outer tube 606 may be extruded, molded, or otherwise integrally formed with the inner tube 602. In still other examples, the spiraled outer tube 606 may be formed as a removable sleeve that can be slid on and off the inner tube 602.

The outer lumen 604 may be selectively pressurized with a fluid (e.g., a compliance-adjustment pressurized gas) in a substantially similar manner as the outer lumen of the dynamic compliance circuits 200, 300 discussed above with reference to FIGS. 2-3. The pressure of the fluid within the outer lumen 608 counteracts the pressure from the pressure from the breathing gases in the inner lumen 604. Accordingly, the effective compliance of the inner tube 602 may be dynamically adjusted by controlling the pressure within the outer lumen 608. The pressure within the outer lumen 608 may be adjusted as discussed herein, such as based on pressure of the breathing gases and/or the type of ventilation mode. In examples, where the outer tube 606 is bonded or otherwise fixed to the inner tube 602, the spiral wrap of the outer tube also resists longitudinal expansion of the inner tube 602. With the outer lumen 608 spiraled around the inner tube, higher pressures within the outer lumen 608 (e.g., at least 150%, 200%, 300%, etc. of the pressures within the inner lumen 604) may also provide the benefit of high radial strength or resistance to expansion while still allowing for bending to connecting the tubing to the patient.

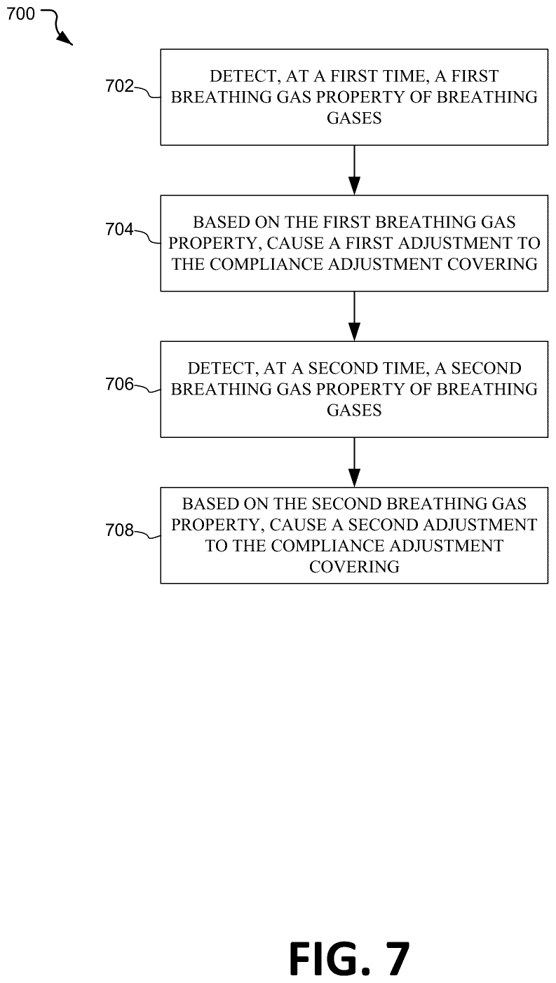

FIG. 7 depicts an example method 700 for dynamically adjusting an effective compliance of an inner tube, such as an inspiratory tube, of a dynamic compliance circuit that includes a compliance adjustment covering. The example methods include operations that may be implemented or performed by the systems and devices disclosed herein. For example, the operations may be performed by a medical ventilator and/or a control system for a dynamic compliance circuit. In various implementations, the control system may be a standalone control system or integrated into the control system of another device. For instance, the control system for the dynamic compliance circuit may be integrated into a control system for inflating a cuff of an endotracheal tube.

At operation 702, at a first time, a first breathing gas property of breathing gases flowing through the inner tube (e.g., through the inner lumen of an inspiratory tube) is detected. The first breathing gas property may be a pressure and/or flow of the breathing gases, and the first breathing gas property may detected by a gas property sensor, such as a pressure sensor or a flow sensor.

At operation 704, based on the first breathing gas property, a first adjustment to the compliance adjustment covering is made. The first adjustment to the compliance adjustment covering may be to increase the effective compliance of the inner tube.

The ventilator, or control system for the dynamic compliance circuit, causes the adjustment by activating a motor or compressor depending on the type of compliance adjustment covering that is used in the dynamic breathing circuit. For example, where the compliance adjustment covering includes an outer lumen that can be pressurized, causing the adjustment may include activating a compressor and/or controlling a valve to adjust the pressure of the fluid within the outer lumen. Where the first gas property is a pressure, the pressure of the compliance-adjusting pressurized gas may be adjusted to be at least the pressure of the first gas property, or other pressures described herein. In examples, where the compliance adjustment covering includes a mesh cover that can be tightened by tightening extensions, causing the adjustment may include activating a motor to pull or loosen the tightening extension.

In other examples, the first adjustment to the compliance adjustment covering may be proactive in that the adjustment to the dynamic compliance circuit increases prior to the increase in breathing gas pressure in the inner tube. For instance, depending on the mode of ventilation, the timing of inhalation phases or increase in breathing gas pressure is set or known. As such, the ventilator may communicate the timing of the increase in breathing gas pressure is communicated to the control system or module for the dynamic compliance circuit. The dynamic compliance circuit may then be adjusted prior to the increase in breathing gas pressure such that the dynamic compliance circuit is prepared for the increase in breathing gas pressure.

At operation 706, at a second time, a second breathing gas property of breathing gases flowing through the inner tube (e.g., through the inner lumen of an inspiratory tube) is detected. The second breathing gas property may be the same type of property as the first gas property. For example, the first breathing gas property may be a first pressure of the breathing gases and the second breathing gas property may be a second pressure of the breathing gases.

At operation 708, based on second breathing gas property, a second adjustment to the compliance adjustment covering is made. The second adjustment to the compliance adjustment covering may be to decrease the effective compliance of the inner tube. For instance, in an example where the second breathing gas property indicates a lower breathing gas pressure than the first breathing gas property, the second adjustment to the compliance adjustment covering may be to reduce the effective compliance of the inner tube. For example, pressure of the compliance-adjusting pressurized gas may be released and/or tension on the tightening extension may be released. Accordingly, instead of switching out a patient circuit upon changes in pressure, the compliance of the patient circuit or inspiratory tube can be dynamically adjusted based on the properties of the breathing gases flowing through the tube.

In some examples, the adjustments to the compliance adjustment covering may be made relatively quickly, such as in the same breath or phase of a breath (such as an inhalation phase or an exhalation phase). For instance, the adjustments may be performed within less than about 5 seconds from one another. In other examples, the compliance adjustment covering may be adjusted at different intervals, such as upon a change in ventilation mode.

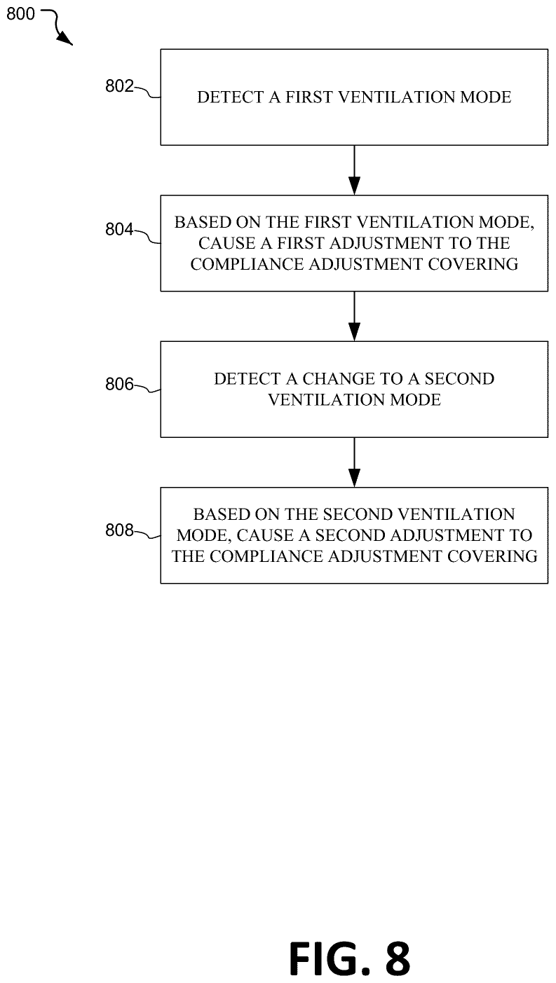

FIG. 8 depicts another example method 800 for dynamically adjusting an effective compliance of an inner tube, such as an inspiratory tube, of a dynamic compliance circuit that includes a compliance adjustment covering. At operation 802, a first ventilation mode is detected. The detection of the ventilation mode may be detected by receiving an indication from the ventilator of the active ventilation mode. In other examples, detection of the ventilation mode may be performed through gas properties of the breathing gases, which may be detected or sensed by one or more gas property sensors. At operation 804, based on the first ventilation mode, a first adjustment to the compliance adjustment covering is made. The adjustment may be the types of adjustments discussed herein. As an example, where the first ventilation mode is a traditional ventilation mode, little to no adjustment may be made to the compliance adjustment covering as the standard or inherent compliance of the inner tube is sufficient to resist significant expansion.

At operation 806, a change to a second ventilation mode is detected. The change to the second ventilation mode may be detected from an indication provided by the ventilator and/or based on a change in gas properties of the breathing gases flowing through the inner tube. At operation 808, based on the second ventilation mode, a second adjustment to the compliance adjustment covering is made. As an example, where the second ventilation mode is a high-pressure and/or high-frequency ventilation mode, such as HFOV, the second adjustment is to increase the effective compliance of the inner tube, such as by increasing the pressure of fluids in an outer lumen or causing a pulling or tightening of a tightening extension.

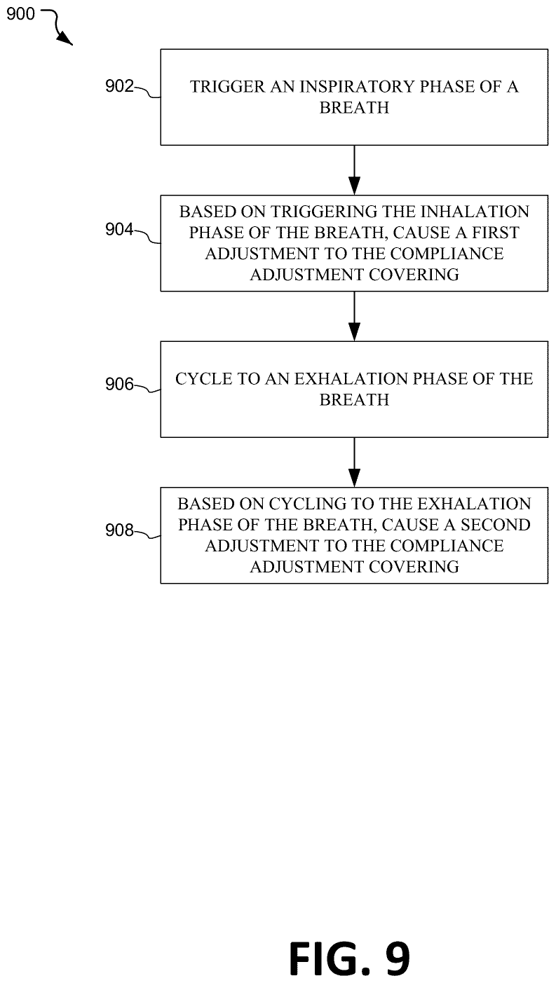

FIG. 9 depicts another example method 900 for dynamically adjusting an effective compliance of an inner tube, such as an inspiratory tube, of a dynamic compliance circuit that includes a compliance adjustment covering. At operation 902, the ventilator triggers an inhalation phase of a breath to deliver breathing gases. At operation 904, based on triggering the inhalation phase of the breath, the ventilator causes a first adjustment to the compliance adjustment covering. Because the inhalation phase of the breath increases the pressure of the breathing gases in the inner tubing, the first adjustment to the compliance adjustment covering is to increase the effective compliance of the inner tube to resist the expansion of the inner tube.

At operation 906, the ventilator cycles to an exhalation phase of the breath. At operation 908, based on cycling to the exhalation phase, the ventilator causes a second adjustment to the compliance adjustment covering. Because the exhalation phase may produce lower breathing gas pressures than the inhalation phase, the second adjustment to the compliance adjustment covering may reduce the effective compliance of the compliance adjustment covering. The method 900 may repeat for each breath.

Although the present disclosure discusses the implementation of these techniques in the context of a ventilator capable of performing dynamic adjustments to the compliance of a patient circuit, the techniques introduced above may be implemented for a variety of medical devices or devices utilizing flow sensors. A person of skill in the art will understand that the technology described in the context of a medical ventilator for human patients could be adapted for use with other systems such as ventilators for non-human patients or general gas transport systems. Additionally, a person of ordinary skill in the art will understand that the modeled exhalation flow may be implemented in a variety of breathing circuit setups.

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A medical ventilation system, comprising:
a dynamic compliance circuit comprising:
an inspiratory tube extending from an inhalation port of a medical ventilator, the inspiratory tube defining an inner lumen for carrying breathing gases from the inhalation port towards a patient; and
a compliance adjustment covering coupled to the inspiratory tube, wherein adjustments to the compliance adjustment covering alter an effective compliance of the inspiratory tube;
a processor; and
memory storing instructions that, when executed by the processor, cause the system to perform operations including, based a type of ventilation mode of the ventilator, causing an adjustment to the compliance adjustment covering to alter the effective compliance of the inspiratory tube.

2. The medical ventilation system of claim 1, wherein the compliance adjustment covering includes a mesh capable of being tightened by pulling tightening extensions extending from the mesh.

3. The medical ventilation system of claim 2, wherein causing the adjustment to the compliance adjustment covering includes causing the tightening extensions to be pulled.

4. The medical ventilation system of claim 1, wherein the compliance adjustment covering includes an outer tube defining an outer lumen between the outer tube and inspiratory tube, the outer lumen capable of receiving a compliance-adjusting pressurized gas.

5. The medical ventilation system of claim 4, wherein causing the adjustment to the compliance adjustment covering includes causing a delivery of the compliance-adjusting pressurized gas to increase the effective compliance of the inspiratory tube.

6. The medical ventilation system of claim 4, wherein the outer lumen is at least partially filled with a non-Newtonian fluid in contact with an outer surface of the inspiratory tube.

7. The medical ventilation system of claim 1, wherein the compliance adjustment covering includes an outer tube spirally wrapped around the inspiratory tube, the outer tube defining an outer lumen between the outer tube and inspiratory tube, the outer lumen capable of receiving a compliance-adjusting pressurized gas.

8. A dynamic compliance circuit for medical ventilation comprising:
an inspiratory tube extending from an inhalation port of a medical ventilator, the inspiratory tube having an inner lumen for carrying breathing gases from the inhalation port towards a patient; and
a compliance adjustment covering coupled to the inspiratory tube, wherein adjustments to the compliance adjustment covering alter an effective compliance of the inspiratory tube.

9. The dynamic compliance circuit of claim 8, wherein the compliance adjustment covering includes a mesh capable of being tightened by pulling tightening extensions extending from the mesh.

10. The dynamic compliance circuit of claim 8, wherein the compliance adjustment covering includes an outer tube defining an outer lumen between the outer tube and inspiratory tube, the outer lumen capable of receiving a compliance-adjusting pressurized gas.

11. The dynamic compliance circuit of claim 8, wherein the compliance adjustment covering includes an outer tube spirally wrapped around the inspiratory tube, the outer tube defining an outer lumen between the outer tube and inspiratory tube, the outer lumen capable of receiving a compliance-adjusting pressurized gas.

12. A method for controlling an effective compliance of an inspiratory tube, the method comprising:
detecting at a first time, based on a gas property sensor, a first breathing gas property of breathing gases flowing through the inspiratory tube;
based on the first breathing gas property, causing a first adjustment to the compliance adjustment covering to increase the effective compliance of the inspiratory tube;
detecting at a second time, based on the gas property sensor, a second breathing gas property of breathing gases flowing through the inspiratory tube; and
based on the second breathing gas property, causing a second adjustment to the compliance adjustment covering to decrease the effective compliance of the inspiratory tube.

13. The method of claim 12, wherein:
the first breathing gas property is a first breathing gas pressure;
the second breathing gas property is a second breathing gas pressure; and
the first breathing gas pressure is higher than the second breathing gas pressure.

14. The method of claim 12, wherein the first adjustment and the second adjustment are caused within a time period of less than 5 seconds.

15. The method of claim 12, wherein:
the compliance adjustment covering includes an outer tube defining an outer lumen, the outer lumen capable of receiving a compliance-adjusting pressurized gas; and
causing a first adjustment to the compliance adjustment covering includes increasing the pressure of the compliance-adjusting pressurized gas.
